(19) United States
(12) Patent Application Publication
(10) Pub. No.: US 2008/0233187 A1
(43) Pub. Date: Sep. 25, 2008

(54) Method of Production of Fine-Crystalline Mixture Containing Non-Steroid Anti-Inflammatory Drug, Fine-Crystalline Mixture Obtainable by this Method and Solid Pharmaceutical Composition Containing this Mixture

(76) Inventors: Michal Svoboda, Praha (CZ); Xenia Svobodova, Praha (CZ)

Correspondence Address: NOTARO AND MICHALOS, 100 DUTCH HILL ROAD, SUITE 110, ORANGEBURG, NY, 10962-2100, US

(21) Appl. No.: 12/063,845

(22) PCT Filed: Aug. 10, 2006
(86) PCT No.: PCT/CZ06/00051

§ 371 Date: Feb. 14, 2008

(30) Foreign Application Priority Data
Aug. 30, 2005 (CZ) PV 2005-546

(51) Int. Cl. A61K 31/192; A61K 9/28; A61K 9/48; A61P 29/00

(52) U.S. Cl. 424/456; 514/570; 424/474

(57) ABSTRACT

The invention concerns a method of production of a fine-crystalline mixture containing a non-steroid anti-inflammatory drug and an auxiliary substance, wherein a coarse-crystalline substance from the group of non-steroid anti-inflammatory drugs is dissolved in a solvent at an increased temperature, the solution is subsequently distributed at rapid chilling into a cooling liquid containing the auxiliary substance, said cooling liquid being placed in an ice bath, and the product is then filtered off and dried. It further concerns the fine-crystalline mixture of the non-steroid anti-inflammatory drug and the auxiliary substance that can be obtained by the said method. The invention further concerns a solid pharmaceutical composition, having substantially improved dissolution properties, which contains 60 to 78% w/w of the fine-crystalline mixture, 17 to 40% w/w of microcrystalline cellulose, colloidal silicon dioxide in an amount of up to 0.3% w/w, a disintegrant in an amount of up to 4% w/w and optionally a surface active compound in an amount of up to 0.1% w/w. This solid pharmaceutical composition can be filled into capsules or used for the preparation of tablets.

Method of production of fine-crystalline mixture containing non-steroid anti-inflammatory drug, fine-crystalline mixture obtainable by this method and solid pharmaceutical composition containing this mixture

### FIELD OF THE INVENTION

The invention relates to a method of producing a fine-crystalline mixture containing a non-steroid anti-inflammatory drug and an auxiliary substance, with improved flow properties suitable for filling in capsules or for pressing tablets. The invention also comprises the fine-crystalline mixture that can be prepared by the method of the invention and a solid pharmaceutical preparation containing this finely crystalline mixture.

### BACKGROUND ART

Non-steroid anti-inflammatory drugs, commonly abbreviated as NSAID or referred to as non-steroid anti-rheumatics (NSA) for their main indication in the treatment of rheumatic disorders, are of a considerable clinical importance. They are widely used because of their antipyretic, antiflogistic and analgesic effects. Inhibition of the synthesis of prostaglandins by means of inhibition of the enzyme of their synthesis, cyclooxygenase 1 and 2 (COX1 and COX2; Vane: Nat. New Biol.: 231(25), 232-5, 1971) is the basic mechanism of action of these compounds. Chemically they represent a heterogeneous group of compounds showing common pharmacological properties.

Although NSAs definitely inhibit the synthesis of prostaglandins, they possess also other pharmacological properties that can contribute to their therapeutical effectiveness. Some of them (diclofenac, indometacine) inhibit the enzyme lipooxygenase, thereby lowering the production of leucotriens by leukocytes and synovial cells. Others (piroxicam) inhibit the production of hydrogen peroxide in activated neutrophils. Some NSAs can interfere with the synthesis of proteoglycans in chondrocytes (ASA, indometacine), with the transmembrane ion transfer, with intercellular structures. Although these “non-prostaglandin” effects of the NSAs display seldom an effect in humans, they can explain the frequently observed variability of the therapeutical response.

The analgesic activity of non-steroid anti-inflammatory drugs (at moderate and intermediate intensity of pain) is independent of anti-inflammatory properties.

The antipyretic effect, that can be observed for all non-steroid anti-inflammatory drugs, is mediated by their action in the thermoregulatory centre in the hypothalamus.

Nimesulide, the systematic chemical name of which is N-(4-nitro-2-phenoxyphenyl)-methanesulphonamide, piroxicam, the chemical name of which is 4-hydroxy-2-methyl-3-[(2-pyridyl)aminocarbonyl]-2H-1,2-benzothiazin-1,1-dioxide and ibuprofen, the chemical name of which is 2-(4-isobutylphenyl)-propionic acid, belong among frequently used drugs of this group.

Nimesulide is used particularly in the long-term treatment of osteoarthritis (Huskisson; Clin. Exp. Rheumatol. 19 (suppl. 22), S21-25, 2001) and shows also neuroprotective activity in the brain (Candelario-Jalil et al.; Eur. J. Pharmacol. 453, 189-195, 2002). Its undesirable effects in the liver are being studied (Dumortier et al.: Gastroenterol. Clin. Biol. 26, 415-416, 2002).

Piroxicam is used as antirheumatic and antiflogistic and aside from this it is used also as analgesic, particularly in surgical operations in pediatry (Dix et al.: Anaesthesia 59(10), 984-7, 2004).

Ibuprofen, like other NSAs based on the 2-arylpropionic acid, contains a single chiral centre at an asymmetrically substituted carbon atom, and therefore exists in two enantiomeric forms as S(+)-2-(4-isobutylphenyl)-propionic acid or as R(−)-2-(4-isobutylphenyl)-propionic acid. Although ibuprofen has been used for many years in therapy in the racemic form, it is known that the active enantiomer is the S-enantiomer, further referred to as S(+)-ibuprofen (Adams et al, Curr. Med. Res. Opin., 3, 552 (1975) and J. Pharm. Pharmacol. 28, 256-257 (1976).

In contrast to the R(−)-ibuprofen, S(+)-ibuprofen inhibits the cyclooxygenase in clinically relevant concentrations. The enantiomers are, with regard to their pharmacological properties, different substances and they differ also in the metabolic pathways. R(−)-ibuprofen takes part in the processes of the metabolism of lipids and together with endogenous fatty acids is being incorporated into triglycerides. S(+)-ibuprofen does not undergo these less common metabolic reactions and is thus considered to be a metabolically more “pure” form than the racemate. The racemic mixture administered to a human shows the conversion of 50 to 60% of R(−)-enantiomer to the S(+) form, but this inversion differs in different humans, depending also on the state to be influenced. Recent studies have shown that for achieving the clinical effect of the racemic ibuprofen it is sufficient to administer the half of the dose of the pure S(+)-ibuprofen. (A. M. Evans; Clin. Rheumatol. 20, suppl. 1, S9-14, 2001).

Commercially available preparations, containing racemic ibuprofen as the active ingredient, are known in the art, and there are many patents (e.g. U.S. Pat. No. 5,512,302, WO 9410994, U.S. Pat. No. 4,911,921, U.S. Pat. No. 4,806,359, U.S. Pat. No. 4,835,187) dealing with various formulations containing racemic ibuprofen. Since the active substance is racemic ibuprofen, the pharmaceutical preparation must contain a higher amount of the active ingredient to achieve a comparable therapeutic effect, the organism is thus unnecessarily stressed with ballast substances and the pharmaceutical form must be bulkier.

Processes, used for the preparation of pharmaceutical forms containing racemic ibuprofen are not directly transferable to S(+)-ibuprofen, mainly because S(+)-ibuprofen has a substantially lower melting point (50 to 54° C.) compared to the racemate (75 to 78° C.) and different physical properties such as a different behaviour when being dissolved in common solvents. The crystallization of S(+)-ibuprofen in the form of fine crystals is therefore difficult. Large crystals of S(+)-ibuprofen cannot be grinded to smaller particles with regard to the low melting point, easily reached due to the heat rising from the grinding step, and simple crystallization does not lead to the desired result.

The patent DE 3922441 solves this problem by using the calcium salt of S(+)-ibuprofen. Analogously, the patent applications WO 92/20334 and WO 94/10994 describe pharmaceutical compositions containing S(+)-ibuprofen in the form of a salt, particularly the sodium salt. Apparently, the use of S(+)-ibuprofen in the form of a salt allows an increase of the melting point and avoids the problems particularly connected with the low melting point of the active ingredient. To the S(+)-ibuprofen in the form of a salt additional components, such as diuretics (WO 92/05786) or antihistaminics (WO 92/05783), can be optionally added into the pharmaceutical composition.

The U.S. Pat. No. 5,869,101 describes a method of production of S(+)-ibuprofen particles lo having improved flow properties wherein the coarse-crystalline S(+)-ibuprofen in a molten condition is dispersed in a cooling non-solving liquid and is chilled therein, yielding a fine-crystalline primary structure, the product is then filtered and dried in the form of resulting agglomerates as newly-formed secondary product. The thus obtained particles are suitable for direct pressing of tablets and with the addition of auxiliary substances also for the production of tablets having a retarded release of the active substances. The disadvantage of this process is that it requires a high-quality dispersing apparatus allowing very high speed of rotation (rpm) for stirring the cooling liquid, which is necessary for yielding particles small enough of the agglomerate.

A necessity to develop a method of production of non-steroid anti-inflammatory drugs, particularly S(+)-ibuprofen, in the form suitable for filling in capsules or pressing of tablets, without using an undesirable amount of pharmaceutically acceptable auxiliary substances and retaining only low requirements on the instrumentation equipment, is apparent from the above mentioned facts.

It was found that the compounds from the group of non-steroid anti-inflammatory drugs can be obtained in the form particularly suitable for the use in pharmaceutical preparations by the method of the present invention.

It was also found that a solid pharmaceutical composition obtained using the product prepared by the method of the present invention shows a surprisingly improved release of the active ingredient, compared for instance to the commercially available pharmaceutical composition containing S(+)-ibuprofen.

### DESCRIPTION OF THE INVENTION

An object of the present invention is a method of production of a fine-crystalline mixture containing a non-steroid anti-inflammatory drug and an auxiliary substance, wherein a coarse-crystalline substance from the group of non-steroid anti-inflammatory drugs is dissolved in a solvent at an increased temperature, the solution is subsequently distributed at rapid chilling into a cooling liquid containing the auxiliary substance, said cooling liquid being placed in an ice bath, and the product is then filtered off and dried.

The coarse-crystalline substance from the group of non-steroid anti-inflammatory drugs for performing the method of the invention is preferably S(+)-ibuprofen.

Water is preferably used as the cooling liquid.

It is an aspect of the present invention that the auxiliary substance is selected from the group containing microcrystalline cellulose, silicon dioxide and polyvinylpyrrolidone, preferably the auxiliary substance is microcrystalline cellulose.

The presence of the auxiliary substance in the cooling liquid facilitates the formation of crystals of suitable size and flow properties for the formulation in the form of tablets or for filling in capsules. As suitable auxiliary substances for the crystallization, e.g. polyvinylpyrrolidone (kollidon 30), microcrystalline cellulose Avicel® (registered trademark of FMC Corporation), colloidal silicon dioxide Aerosil® 200 (registered trademark of Degussa A.G.) were used

It is a further aspect of the present invention that the solvent is selected from the group containing ketones, lower alcohols and carboxylic acids. Preferably, the solvent is selected from the group containing acetone, ethylmethylketone, 2-propanol and acetic acid.

The solvent for dissolving the coarse-crystalline substance from the group of non-steroid anti-inflammatory drugs must be non-reactive towards the said substance, miscible with water and non-toxic. All solvents listed above belong to the hazard rating of class 3. This class covers the solvents that are not dangerous to the human health and thus are acceptable in medicaments with the daily limit of 50 mg or less or up to 0.5% w/w (see Residual Solvents in Marketed Products, Recommendation for implementation in the member states, Europe's Committee for Proprietary Medicinal Products, July 1999).

As another aspect of the present invention the coarse-crystalline substance from the group of non-steroid anti-inflammatory drugs is dissolved in the solvent at the temperature range of from 35° C. up to the boiling point of the solvent preferably at a temperature range of from 48 to 55° C.

A further object of the present invention is a fine-crystalline mixture containing the non-steroid anti-inflammatory drug and the auxiliary substance, preparable by the method of the invention.

It is an aspect of the present invention that the fine-crystalline mixture of the present invention contains 20 to 99.5% w/w of the non-steroid anti-inflammatory drug and 0.5 to 80% w/w of the auxiliary substance.

In a preferred embodiment, the fine-crystalline mixture of the present invention contains 76.9 to 99.5% w/w of the non-steroid anti-inflammatory drug and 0.5 to 23.1% w/w of the auxiliary substance.

The non-steroid anti-inflammatory drug in accordance with the invention is preferably S(+)-ibuprofen.

In a further aspect of the invention the fine-crystalline mixture of the invention contains the auxiliary substance selected from the group containing microcrystalline cellulose, silicon dioxide or polyvinylpyrrolidone. In a preferred embodiment, the auxiliary substance is microcrystalline cellulose.

The purity of the fine-crystalline mixture thus obtained substantially depends on the content of the S(+)-isomer in the starting coarse-crystalline, commercially available mixture. Such mixture has e.g. in case of using ibuprofen the declared minimum content of S(+)-ibuprofen 98% w/w, whereas the impurity is R(−)-ibuprofen. The minor contents of R(−)-ibuprofen in the fine-crystalline mixture of the invention does not influence negatively its properties, preparation or use.

Another object of the present invention is a solid pharmaceutical composition, containing 60 to 78% w/w of the fine-crystalline mixture of the invention, 17 to 40% w/w of microcrystalline cellulose, colloidal silicon dioxide in an amount of up to 0.3% w/w, a disintegrant in an amount of up to 4% w/w and optionally a surfactant in an amount of up to 0.1% w/w.

It is an aspect of the invention that the pharmaceutical composition contains a compound selected from the group comprising sodium carboxymethylstarch and crosscarmelose sodium as a disintegrant.

The surfactant is preferably sodium laurylsulphate.

In a preferred embodiment, the solid pharmaceutical product of the invention contains the fine-crystalline mixture of the invention, wherein the non-steroid anti-inflammatory drug comprised in the fine-crystalline mixture of the invention, which is a component of the solid pharmaceutical composition, is S(+)-ibuprofen and the auxiliary substance is the compound selected from the group containing microcrystalline cellulose, silicon dioxide or polyvinylpyrrolidone, preferably microcrystalline cellulose.

The solid pharmaceutical composition of the present invention can be prepared by direct mixing of the components and shows good flow properties, compressibility and pressability. The fine-crystalline mixture namely consists of irregularly shaped crystals of not excessively pronounced prolonged shape, the length to width ratio of which is about 2:1; that makes the obtained product particularly suitable for the preparation of tablets by direct tabletting and for filling into gelatine capsules. This composition shows substantially improved release rate of the active ingredient from both mentioned pharmaceutical forms in comparison with a composition containing the coarse-crystalline non-steroid anti-inflammatory drug.

The solid pharmaceutical composition of the invention can be filled into gelatine capsules or can be used for the preparation of tablets. The tablets can be prepared by direct pressing of the solid pharmaceutical composition of the invention and they can be optionally coated or film-coated by conventional methods.

### FIGURES

FIG. 1 shows the release rate of the active ingredient from a capsule manufactured in accordance with recipe A, i.e. containing the fine-crystalline mixture containing S(+)-ibuprofen and the auxiliary substance microcrystalline cellulose having the size of particles 100 μm (Avicel®) and the release rate of the active ingredient from a capsule containing the coarse-crystalline S(+)-ibuprofen in the same ratio to Avicel®. At the ordinate (y-axis) the amount of the released active ingredient is outlined as %, and at the abscissa (x-axis) the time is outlined as minutes.

FIG. 2 shows the release rate of the active ingredient from a capsule manufactured in accordance with recipe A, whereby the ratio of S(+)-ibuprofen and microcrystalline cellulose (Avicel®), used for the preparation of the fine-crystalline mixture, was varied. The weight ratios of S(+)-ibuprofen and Avicel® used were (in grams): 10:6, 10:3, 10:1.5. At the ordinate (y-axis) the amount of the released active ingredient is outlined as %, and at the abscissa (x-axis) the time is outlined as minutes.

FIG. 3 shows the release rate of the active ingredient from a tablet manufactured in accordance with recipe C1 and from a commercially available tablet containing S(+)-ibuprofen and a disintegrant (Seractil®); at the ordinate (y-axis) the amount of the released active ingredient is outlined as %, and at the abscissa (x-axis) the time is outlined as minutes.

FIG. 4 shows the release rate of the active ingredient from capsules manufactured in accordance with recipe C2. The S(+)-ibuprofen used was obtained using acetone (4A), 2-propanol (4B), methylethylketone (4C) and acetic acid (4D) as the solvent.

FIG. 4E shows the release rate of the active ingredient from capsules, containing racemic ibuprofen as the active ingredient, obtained by the method of the invention from the coarse-crystalline racemic ibuprofen.

FIG. 5 shows the release rate of the active ingredient from a tablet manufactured in accordance with recipe C2, whereas nimesulide was used as the active ingredient. At the ordinate (y-axis) the amount of the released active ingredient is outlined as %, and at the abscissa (x-axis) the time is outlined as minutes.

FIG. 6 shows the release rate of the active ingredient from a tablet manufactured in accordance with recipe C2, whereas piroxicam was used as the active ingredient. At the ordinate (y-axis) the amount of the released active ingredient is outlined as %, and at the abscissa (x-axis) the time is outlined as minutes.

FIG. 7 shows the photographs of the crystals of the fine-crystalline mixture containing S(+)-ibuprofen, obtained by the method of the present invention using acetone (7A), 2-propanol (7B), ethylmethylketone (7C) and acetic acid (7D) as the solvents, of the racemic mixture of ibuprofen obtained by the method of the invention (7E) and of the coarse-crystalline S(+)-ibuprofen (7F).

FIG. 8 shows the photographs of the crystals of the fine-crystalline mixture containing piroxicam, obtained by the method of the present invention, using acetone as the solvent and of the crystals of the starting coarse-crystalline piroxicam.

### EXAMPLES

### Example 1

94.3 g of acetone is heated to 50° C. by means of a water bath. After reaching this temperature, 120 g of coarse-crystalline S(+)-ibuprofen is added and the mixture is shortly stirred at 50° C. 480 g of cold water having room temperature (about 20° C.) is poured into a suitable vessel, then 36 g of microcrystalline cellulose is added and the mixture is stirred by means of a magnetic stirrer. The vessel containing water and microcrystalline cellulose is put in an ice bath.

The dissolved S(+)-ibuprofen in acetone is added by pouring into the water with microcrystalline cellulose under continuous stirring at a speed over 800 rpm. After yielding of a solid product, said product is separated by means of a suitable filter and washed with cold water. The final product is dried in a lyophilisator.

### Example 2

7.9 g of acetone is heated to 50° C. by means of a water bath. After reaching this temperature, 10 g of coarse-crystalline S(+)-ibuprofen is added and the mixture is shortly stirred at 50° C. 40 g of cold water (20° C.) is poured into a suitable vessel, 0.055 g of polyvinylpyrrolidone (kollidon 30) is added and the mixture is stirred by means of a magnetic stirrer. The vessel containing water and polyvinylpyrrolidone is put in an ice bath.

The S(+)-ibuprofen dissolved in acetone is added by pouring into the water with polyvinylpyrrolidone under continuous stirring at a speed over 800 rpm. After yielding of a solid product, said product is separated by means of a suitable filter and washed with cold water. The final product is dried in a lyophilisator.

### Example 3

7.9 g of acetone is heated to 50° C. by means of a water bath. After reaching this temperature, 10 g of coarse-crystalline S(+)-ibuprofen is added and the mixture is shortly stirred at 50° C. 40 g of cold water (20° C.) is poured into a suitable vessel, then 0.14 g of sodium laurylsulphate is added and the mixture is stirred by means of a magnetic stirrer. The vessel containing water and sodium laurylsulphate is put in an ice bath.

The S(+)-ibuprofen dissolved in acetone is added by pouring into the water with sodium laurylsulphate under continuous stirring at a speed of over 800 rpm . After yielding of a solid product, said product is separated by means of a suitable filter and washed with cold water. The final product is dried in a lyophilisator.

### Example 4

7.9 g of acetone is heated to 50° C. by means of a water bath. After reaching this temperature, 10 g of coarse-crystalline S(+)-ibuprofen is added and the mixture is shortly stirred at 50° C. 40 g of cold water (20° C.) is poured into a suitable vessel, 1 g of silicon dioxide (aerosil) is added and the mixture is stirred by means of a magnetic stirrer. The vessel containing water and aerosil is put in an ice bath.

The S(+)-ibuprofen dissolved in acetone is added by pouring into the water with aerosol under continuous stirring at a speed of over 800 rpm. After yielding of a solid product, said product is separated using a suitable filter and washed with cold water. The final product is dried in a lyophilisator.

Exemplary recipes for the preparation of a solid pharmaceutical composition for filling in a capsule made of hard gelatine, said solid pharmaceutical composition containing as active substance the fine-crystalline mixture containing S(+)-ibuprofen that was prepared according to the above-listed examples.

The following components are mixed:

### Recipe A:

| Component | % w/w |
|---|---|
| Fine-crystalline mixture containing S(+)-ibuprofen | 78.0 |
| prepared according to Example 1 | |
| Microcrystalline cellulose | 20.2 |
| Colloidal silicon dioxide | 0.3 |
| Magnesium stearate | 1.45 |
| Sodium laurylsulphate | 0.05 |

### Recipe B:

| Component | % w/w |
|---|---|
| Fine-crystalline mixture containing S(+)-ibuprofen | 78.0 |
| prepared according to Example 1 | |
| Microcrystalline cellulose | 17.7 |
| Colloidal silicon dioxide | 0.3 |
| Sodium carboxymethylstarch | 4.0 |

### Recipe C1:

| Component | % w/w |
|---|---|
| Fine-crystalline mixture containing S(+)-ibuprofen | 78.0 |
| prepared according to Example 1 | |
| Microcrystalline cellulose | 17.7 |
| Colloidal silicon dioxide | 0.3 |
| Crosscarmelose sodium | 4.0 |

### Recipe C2:

| Component | % w/w |
|---|---|
| Fine-crystalline mixture containing S(+)-ibuprofen | 69.44 |
| prepared according to Example 1 | |
| Microcrystalline cellulose | 26.70 |
| Colloidal silicon dioxide | 0.30 |
| Crosscarmelose sodium | 3.56 |

### Recipe D:

| Component | % w/w |
|---|---|
| Fine-crystalline mixture containing S(+)-ibuprofen | 60.1 |
| prepared according to Example 2 | |
| Microcrystalline cellulose | 38.1 |
| Colloidal silicon dioxide | 0.3 |
| Magnesium stearate | 1.45 |
| Sodium laurylsulphate | 0.05 |

### Recipe E:

| Component | % w/w |
|---|---|
| Fine-crystalline mixture containing S(+)-ibuprofen | 60.3 |
| prepared according to Example 3 | |
| Microcrystalline cellulose | 37.9 |
| Colloidal silicon dioxide | 0.3 |
| Magnesium stearate | 1.45 |
| Sodium laurylsulphate | 0.05 |

### Recipe F:

| Component | % w/w |
|---|---|
| Fine-crystalline mixture containing S(+)-ibuprofen | 65.8 |
| prepared according to Example 4 | |
| Microcrystalline cellulose | 32.4 |
| Colloidal silicon dioxide | 0.3 |
| Magnesium stearate | 1.45 |
| Sodium laurylsulphate | 0.05 |

The pharmaceutical compositions of the present invention are prepared by direct mixing of the components in the form of a powder and show excellent flow properties.

### Pharmaceutical Form: Capsules

For each capsule, 500 mg of the pharmaceutical composition of the above-mentioned composition were used, said composition in the cases A to C1 and D to F corresponds to 300 mg of S(+)-ibuprofen. The composition of the mixture C2 was designed for tablets having the weight 570 mg, therefore the content of S(+)-ibuprofen in a capsule filled with 500 mg of the mixture was lower, only 266.8 mg. The capsules were filled using conventional laboratory equipment.

The release rate of the active ingredient from the capsules prepared by the process described above, and for comparison also from the tablets, was tested by the method of the European Pharmacopoeia for testing solid pharmaceutical forms (chapter 2.9.3.). The medium used was phosphate buffer having pH 7.2. The results are shown in FIG. 1 to 4.

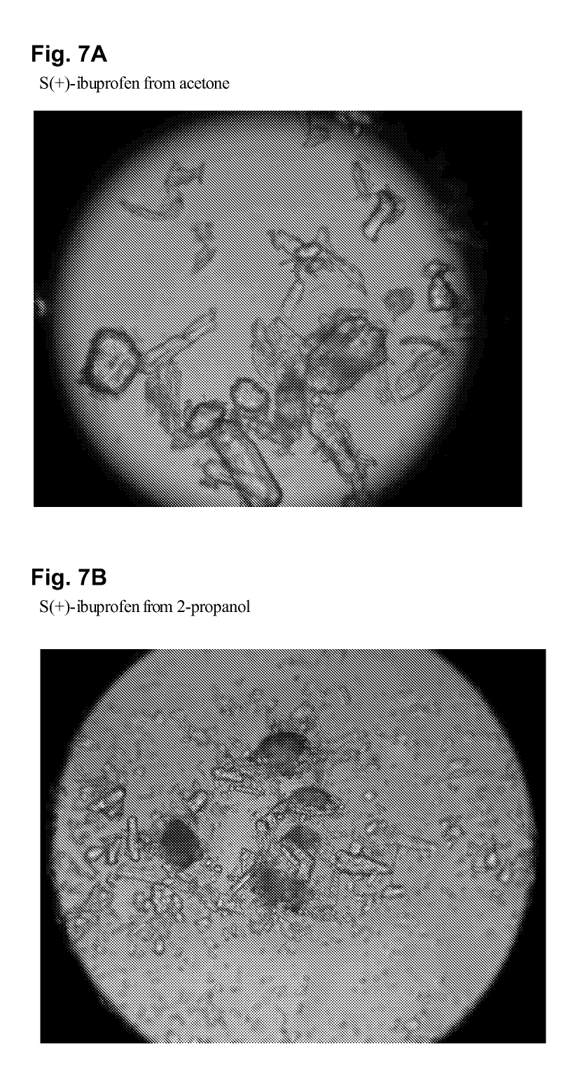

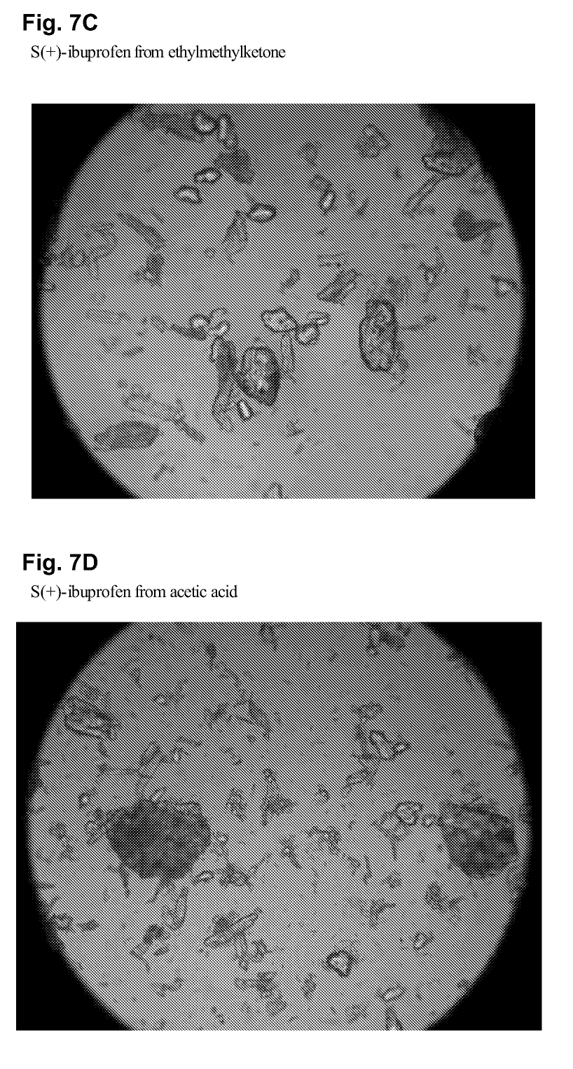

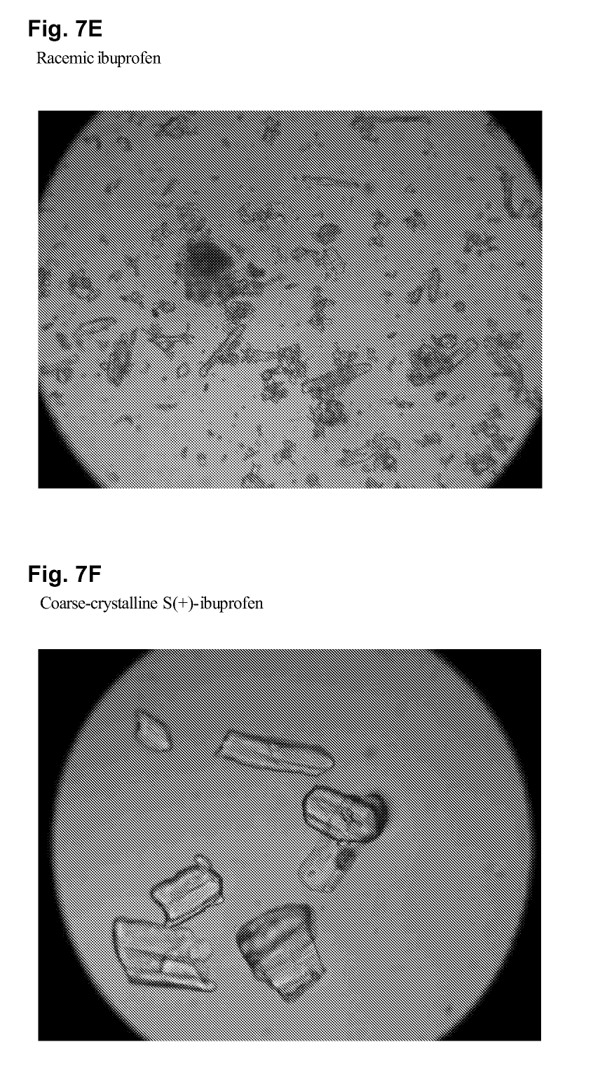

The course of the release of the active ingredient S(+)-ibuprofen from the hard gelatine capsule containing the pharmaceutical composition of the recipe A, i.e. containing the fine-crystalline mixture containing S(+)-ibuprofen and the auxiliary substance Avicel®, shows a substantially better release rate of the active ingredient in comparison with the results obtained when using the common coarse-crystalline S(+)-ibuprofen (FIG. 1), having the lengthwise size of the stick-form crystals about 200 micrometers (see the photograph on FIG. 7). When using the coarse-crystalline S(+)-ibuprofen, the composition of the filling mixture corresponded to the recipe A too, whereas 60% w/w of the active ingredient and 38,2% w/w of microcrystalline cellulose was used (in order to preserve the ratio of the two substances, when microcrystalline cellulose was not used for the isolation of the crystalline form of S(+)-ibuprofen).

In addition to the microcrystalline cellulose (Ex. 1), also polyvinylpyrrolidone, sodium laurylsulphate and silicon dioxide (Ex. 2, 3, and 4) were used as auxiliary substances for the preparation of the fine-crystalline mixture. The best release rate of the active ingredient from the capsules, prepared in accordance with the same recipe, was achieved when using microcrystalline cellulose as the auxiliary substance (results not shown), thus the microcrystalline cellulose was exclusively used as the auxiliary substance in further testing.

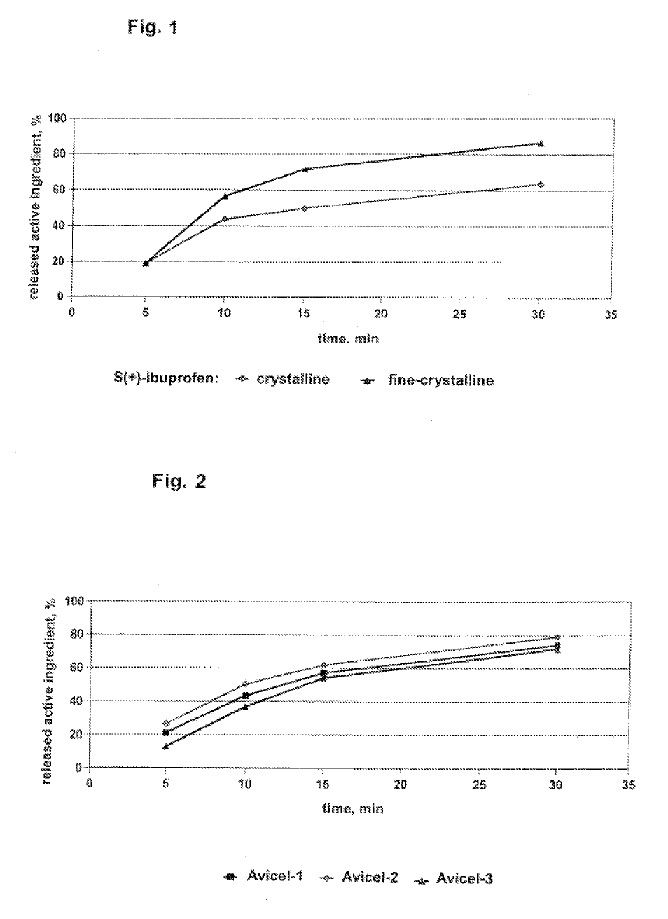

The release rate of the active ingredient depending upon the weight ratio of the active ingredient and microcrystalline cellulose is shown in FIG. 2. The weight ratios of S(+)-ibuprofen and Avicel® used were (in grams): 10:6; 10:3; 10:1.5. From the Figure it may be seen, that the dissolution profile is not substantially influenced by different ratios of the active ingredient to the auxiliary substance.

### Pharmaceutical Form: Tablets

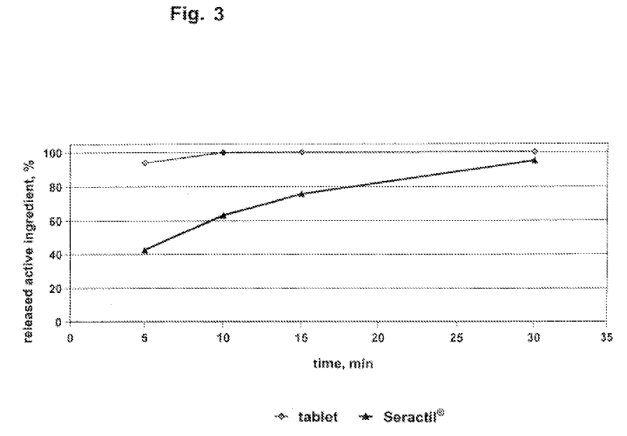

For the manufacture of tablets, the fine-crystalline mixture containing microcrystalline cellulose, prepared in accordance with Example 1, was used. A lentil-shaped tablet had the diameter 13 mm and the height 7 mm, its weight was 500 mg and it was prepared by direct pressing. The dissolution profile is shown in FIG. 3. The tablet of the composition according to the recipe C1, containing a disintegrant, disintegrated very fast. The release rate of the active ingredient was substantially faster and generally higher, compared to the Seractil® tablet, the only commercially available composition which contains S(+)-ibuprofen as well and a disintegrant. was crystallized with the auxiliary substance using ethylmethylketone instead of acetone. When the method of the present invention is applied to said compounds, excessive loss of the substance does not occur during the crystallization with the auxiliary substance.

TABLE 3

| | | | theor. | determined |
|---|---|---|---|---|
| | | | content, | content, |
| | | | in % w/w | in % w/w |
| Active | | auxiliary | of the | of the |
| ingredient | Solvent | substance | mixture | mixture |
| Nimesulide | Acetone | Avicel ® | 76.89 | 76.00 |
| Piroxicam | Ethylmethylketone | Avicel ® | 72.50 | 67.54 |

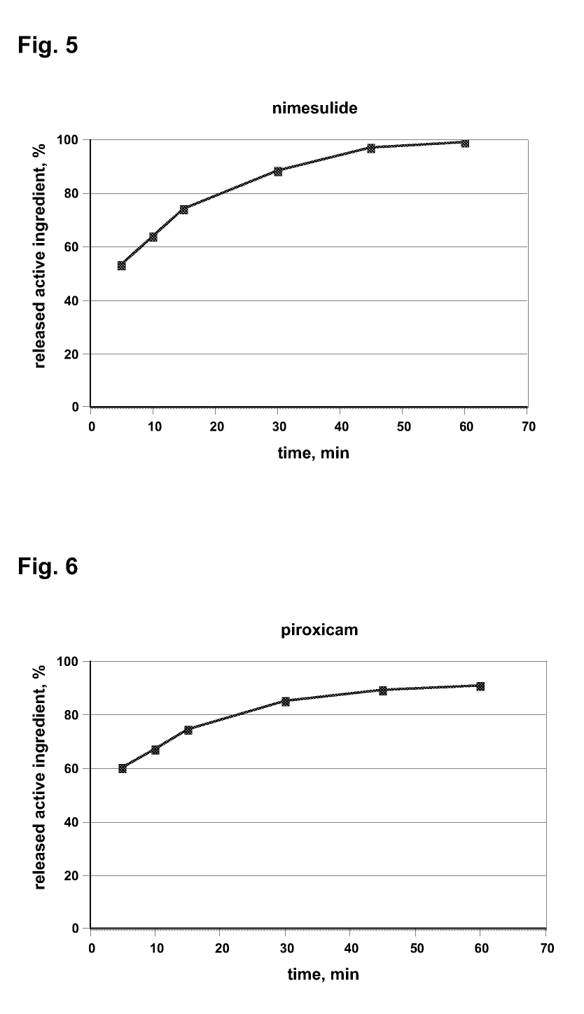

The release rate of the active ingredient nimesulide, as well as piroxicam, was determined in tablets manufactured by using the fine-crystalline mixture of these compounds with microcrystalline cellulose in accordance with the present invention. Acetone was used as a solvent for nimesulide and ethylmethylketone was used for piroxicam. The tablets contained the highest therapeutically acceptable amount of the active ingredient and were manufactured by direct pressing. The nimesulide tablet had the weight of 188 mg (content 100 mg of nimesulide), the diameter of 7 mm and the height of 4 mm. The piroxicam tablet had the weight of 33 mg (content 19 mg of piroxicam), the diameter of 5 mm and the height of 1.5 mm. The dissolution profile of the tablets of the composition of recipe C2, containing the disintegrant, is shown in FIG. 5 and 6. Both substances have shown a very fast release from the tablet form.

In the tests it was verified, that the presence of a glidant, magnesium stearate, is not essential when using the recipe C, i.e. in the presence of the disintegrant, and thus it was not added into the pharmaceutical composition in further testing.

### Alternative Solvents for the Crystallization

In Table 1, the content of S(+)-ibuprofen in the fine-crystalline mixture prepared in accordance with Example 1 is compared, wherein acetone, used in the method of Example 1, was replaced by the solvents shown in Table 1.

TABLE 1

| | | S(+)IBU: | S(+)IBU: |
|---|---|---|---|
| | | calc. | determined |
| | | content, | content, |
| | | in % w/w | in % w/w |
| | Auxiliary | of the | of the |
| solvent | substance | mixture | mixture |
| acetone | Avicel ® | 76.91 | 77.84 |
| 2-propanol | Avicel ® | 76.70 | 77.05 |
| ethylmethylketone | Avicel ® | 77.02 | 76.35 |
| acetic acid | Avicel ® | 76.86 | 77.62 |

The content of the racemic mixture of ibuprofen in the fine-crystalline mixture was 77.90% w/w (calc. content 76.86% w/w) under identical conditions, when using microcrystalline cellulose as the auxiliary substance and acetone as the solvent. These results show that during the crystallization in the presence of the auxiliary substance in any of the solvents tested there is no loss of the active ingredient compared to its calculated, theoretical content.

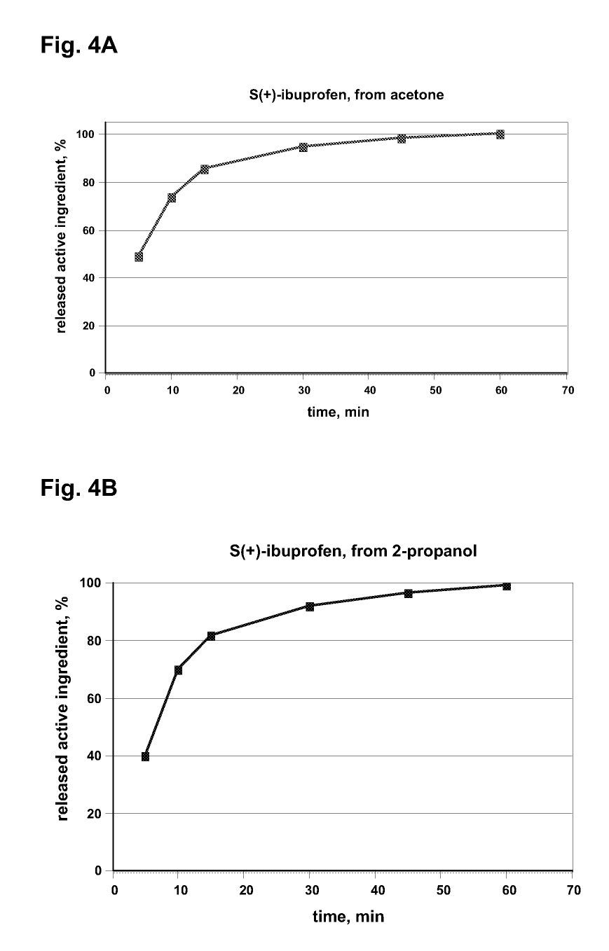

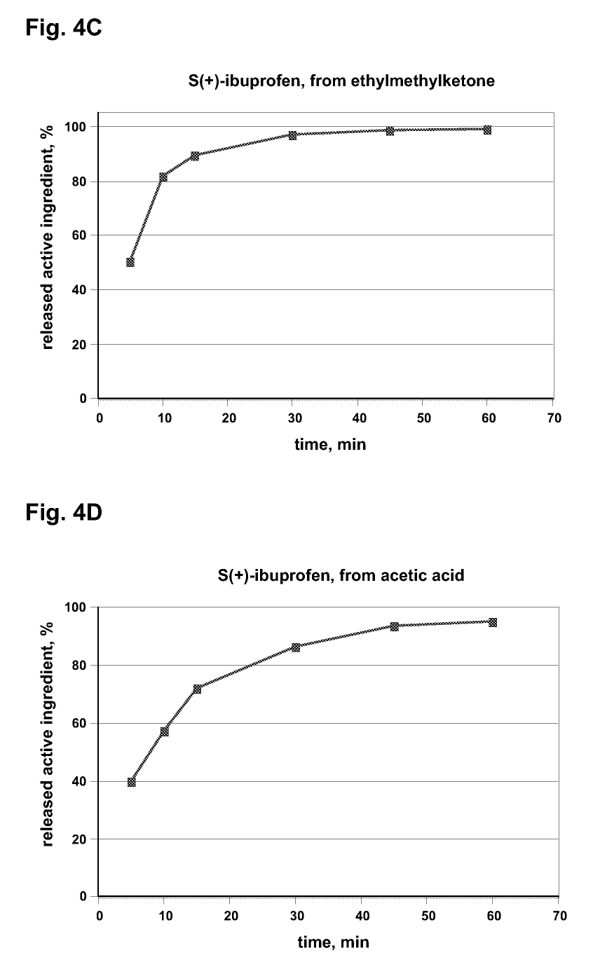

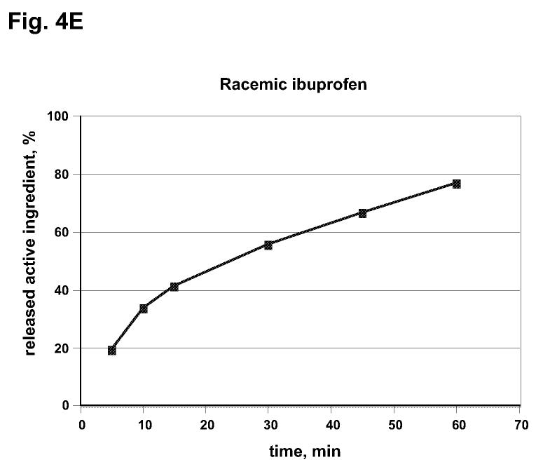

The release rate of the active ingredient S(+)-ibuprofen from the hard gelatine capsule containing the pharmaceutical composition of the composition according to the recipe C2 is shown in FIG. 4. It is compared with the release rate of the analogously prepared racemic ibuprofen, recrystallized from acetone, from an analogical capsule. The fine-crystalline mixture containing S(+)-ibuprofen was prepared with the auxiliary substance Avicel® from the solvents acetone, 2-propanol, ethylmethylketone and acetic acid. The capsules had the weight 500 mg and contained 266.8 mg of the active ingredient. After 30 minutes, the active ingredient in the form of the fine-crystalline mixture was released substantially faster than the ibuprofen racemate in the same form in all cases. The onset (rate) of the S(+)-ibuprofen release has shown the following order depending on the solvent used in the preparation of the fine-crystalline mixture: acetone>ethylmethylketone>2-propanol>>acetic acid.

In Table 2, the most commonly represented sizes of crystals of the fine-crystalline mixture containing S(+)-ibuprofen, obtained using various crystallization solvents, of the fine-crystalline mixture containing racemic ibuprofen and of the coarse-crystalline S(+)-ibuprofen are compared. The auxiliary substance in the fine-crystalline mixtures was Avicel®.

TABLE 2

| | | length of | width of |
|---|---|---|---|
| | | crystals | crystals |
| substance type | solvent | (μm) | (μm) |
| fine-crystalline mixture | acetone | 75 | 20 |
| containing S(+)-ibuprofen | | 35-60 | 25-35 |
| | 2-propanol | 75 | 15 |
| | | 25 | 15 |
| | ethylmethylketone | 45-65 | 15-45 |
| | acetic acid | 25 | 5-15 |
| fine-crystalline racemic | acetone | 145 | 25 |
| mixture | | 20 | 10 |
| coarse-crystalline S(+)- | — | 250 | 65 |
| ibuprofen | | | |

It is obvious from Table 2 that in the fine-crystalline mixture containing S(+)-ibuprofen the crystals became substantially smaller and their shape has changed from a needle-like, prolonged shape to a rounded shape, which contributes to the improved flow properties and pressability of the mixture.

### Other Compounds of the NSAID-type

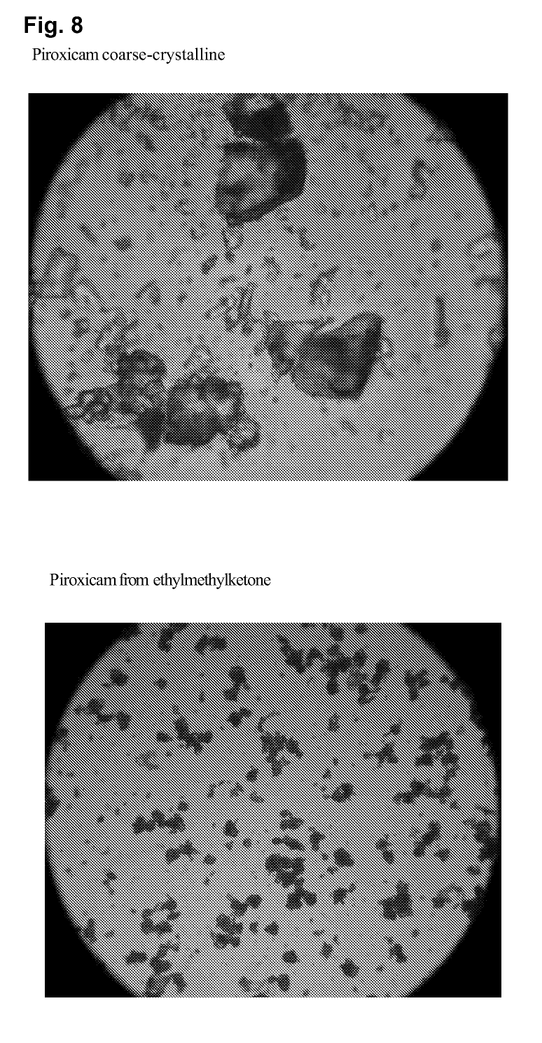

A similar change in the size and shape of crystals occurred after the application of the method of the present invention to other compounds from the group of NSAID, nimesulide and piroxicam (FIG. 8 shows the comparison for piroxicam, similar results were obtained with nimesulide).

In Table 3 there is compared the content of nimesulide or piroxicam respectively in the fine-crystalline mixture prepared by the method of Example 1, whereas piroxicam

1. A method of production of a fine-crystalline mixture containing a non-steroid anti-inflammatory drug and an auxiliary substance, characterized in that a coarse-crystalline substance from the group of non-steroid anti-inflammatory drugs is dissolved in a solvent at an increased temperature, the solution is subsequently distributed at rapid chilling into a cooling liquid containing the auxiliary substance, said cooling liquid being placed in an ice bath, and the product is then filtered of and dried.

2. The method according to claim 1, characterized in that the coarse-crystalline substance from the group of non-steroid anti-inflammatory drugs is S(+)-ibuprofen.

3. The method according to claim 1, characterized in that the cooling liquid is water.

4. The method according to claim 1, characterized in that the auxiliary substance is selected from the group containing microcrystalline cellulose, silicon dioxide and polyvinylpyrrolidone.

5. The method according to claim 4, characterized in that the auxiliary substance is microcrystalline cellulose.

6. The method according to claim 1, characterized in that the solvent is selected from the group containing ketones, lower alcohols and carboxylic acids.

7. The method according to claim 6, characterized in that the solvent is selected from the group containing acetone, ethylmethylketone, 2-propanol and acetic acid.

8. The method according to claim 1, characterized in that the coarse-crystalline substance from the group of non-steroid anti-inflammatory drugs is dissolved in the solvent at a temperature range of from 35° C. up to the boiling temperature of the solvent.

9. The method according to claim 8, characterized in that the coarse-crystalline substance from the group of non-steroid anti-inflammatory drugs is dissolved in the solvent at a temperature range of from 48° C. to 55° C.

10. A fine-crystalline mixture containing the non-steroid anti-inflammatory drug and the auxiliary substance, characterized in that it is preparable by the method according to claim 1.

11. The fine-crystalline mixture according to claim 10, characterized in that it contains 20 to 99.5% w/w of the non-steroid anti-inflammatory drug and 0.5 to 80% w/w of the auxiliary substance.

12. The fine-crystalline mixture according to claim 10, characterized in that it contains 76.9 to 99.5% w/w of the non-steroid anti-inflammatory drug and 0.5 to 23.1% w/w of the auxiliary substance.

13. The fine-crystalline mixture according to claim 10, characterized in that the non-steroid anti-inflammatory drug is S(+)-ibuprofen.

14. The fine-crystalline mixture according to claim 10, characterized in that the auxiliary substance is selected from the group containing microcrystalline cellulose, silicon dioxide or polyvinylpyrrolidone.

15. The fine-crystalline mixture according to claim 1, characterized in that the auxiliary substance is microcrystalline cellulose.

16. A solid pharmaceutical composition, characterized in that it contains 60 to 78% w/w of the fine-crystalline mixture according to claim 10, 17 to 40% w/w of microcrystalline cellulose, colloidal silicon dioxide in an amount of up to 0.3% w/w and a disintegrant in an amount of up to 4% w/w and optionally a surface active compound in the amount of up to 0.1% w/w.

17. The solid pharmaceutical composition according to claim 16, characterized in that the non-steroid anti-inflammatory drug comprised in the fine-crystalline mixture, which is the component of the solid pharmaceutical composition, is S(+)-ibuprofen.

18. The solid pharmaceutical composition according to claim 16, characterized in that the auxiliary substance comprised in the fine-crystalline mixture, which is the component of the solid pharmaceutical composition, is selected from the group containing microcrystalline cellulose, silicon dioxide and polyvinylpyrrolidone.

19. The solid pharmaceutical composition according to claim 18, characterized in that the auxiliary substance is microcrystalline cellulose.

20. The solid pharmaceutical composition according to claim 16, characterized in that the disintegrant is selected from the group containing sodium carboxymethylstarch and croscarmelose sodium.

21. The solid pharmaceutical composition according to claim 16, characterized in that it is in the form of tablets.

22. The solid pharmaceutical composition according to claim 21, characterized in that it is in the form of coated tablets or film-coated tablets.

23. The solid pharmaceutical composition according to claim 16, characterized in that it is filled into gelatine capsules.

24. The solid pharmaceutical composition according to claim 16, characterized in that it contains a surface active compound in an amount of up to 0.1% w/w.

25. The solid pharmaceutical composition according to claim 24, characterized in that the surface active compound is sodium laurylsulphate.

26. The solid pharmaceutical composition according to claim 24, characterized in that it is in the form of tablets.

27. The solid pharmaceutical composition according to claim 26, characterized in that it is in the form of coated tablets or film-coated tablets.

28. The solid pharmaceutical composition according to claim 24, characterized in that it is filled into gelatine capsules.
